(19) United States
(12) Patent Application Publication
(10) Pub. No.: US 2020/0170926 A1
(43) Pub. Date: Jun. 4, 2020

(54) TOPICAL COMPOSITION FOR ENHANCEMENT OF BARRIER FUNCTION

(71) Applicant: Conopco ,Inc., d/b/a UNILEVER, Englewood Cliffs, NJ (US)

(72) Inventors: Rishita CHATTERJEE, Kolkata (IN); Vijay Ramchandra GADGIL, Pune (IN)

(73) Assignee: Conopco, Inc., d/b/a UNILEVER, Englewood Cliffs, NJ (US)

(21) Appl. No.: 16/638,062

(22) PCT Filed: Jul. 5, 2018
(86) PCT No.: PCT/EP2018/068186

§ 371 Date: Feb. 10, 2020

(30) Foreign Application Priority Data
Aug. 17, 2017 (EP) 17186630.4

(51) Int. Cl. A61K 8/97; A61Q 19/08

(52) U.S. Cl. CPC A61K 8/97; A61K 2800/591; A61Q 19/08

(57) ABSTRACT

Disclosed is a topical composition comprising: (i) a first extract comprising botanical actives extracted from Rubia cordifolia; and, (ii) a second extract comprising volatile botanical actives extracted from Camellia sinensis, wherein said second extract comprises E-2-hexenal and linalool at ratio of 0.1:1 to 10:1 parts by weight. The cosmetic composition is useful for upregulation of at N a marker associated with differentiation of keratinocytes to provide epidermal skin barrier benefits.

### FIELD OF THE INVENTION

The invention relates to a topical cosmetic composition which activates certain markers of keratinocyte differentiation, thereby improving the barrier function of skin, particularly that of human skin.

### BACKGROUND OF THE INVENTION

Human skin consists of two important layers, a thicker layer called dermis and a thinner layer on top of it called epidermis. The dermis is responsible for strength, elasticity and thickness. With aging, the thickness of this layer decreases and this is believed to be partially responsible for the appearance of wrinkles in aged skin. The epidermis is composed of a variety of cells which provide resilience and barrier properties. Keratinocytes account for 75 to 80% of the total population of cells in the epidermis. Within the epidermis, the keratinocytes are present in four distinct stages of differentiation. This differentiation is necessary for certain essential functions of the skin, namely for a protective barrier against environmental factors and to prevent loss of water from the body. The skin mainly consists of three main type of cells which are keratinocytes, melanocytes and fibroblasts.

Improper choices of lifestyle, poor dietary habits, hormonal imbalance and exposure to natural and manmade stress-causing factors such as ultraviolet radiation and pollution causes a variety of conditions of which dry skin is most prevalent. Trans-epidermal water loss (TEWL) can be controlled to some extent by application of topical compositions such as moisturizers that prevent or at least reduce it by layering the skin with a hydrating substance.

WO15117957 A1 (Unilever) discloses topical compositions that improve the barrier function of skin. The compositions contain andrographolides which are present in botanical extracts of Andrographis paniculata.

In US2003124159 A (Unilever), barrier function is improved by activation of the nuclear receptor LXRalpha.

US2016166628 A (Unilever) discloses a composition which enhances macro/microcirculation of skin. This composition can be applied to dark circles and underarms. The benefits are achieved through a combination of extracts of Rubia cordifolia and Butea monosperma.

US2006228309 A (Unilever) discloses skin lightening effect of the extracts of Rubia cordifolia.

WO14095198 A1 (Unilever) discloses the use of extracts of tea (Camellia sinensis) in cosmetic compositions for enhancing the immunity of skin.

WO13060710 A2 (Unilever) discloses a topical cosmetic composition that contains extracts of green tea and extracts of black tea at selected ratio for anti-inflammation benefit.

U.S. Pat. No. 5,306,486 A (Unilever, 1994) discloses a cosmetic composition which includes green tea and a sunscreen compound. The composition blocks ultraviolet radiation.

US2005/0084566 A1 (Bavan) discloses a method of the production of instant tea soluble in hot water, comprising the steps of: (a) forming an extract by treating black tea leaves with hard warm water, (b) stripping the extract of its aroma volatiles by passing the tea extract through a flash evaporator under partial vacuum, (c) separating at least 12 wt % as insoluble solids from the extract by subjecting the extract to repeated clarification and polishing to obtain clarified concentrate (d) separating 6 to 10 percent soluble solids from the clarified concentrate, (e) adjusting pH of the concentrate to neutral by adding an edible acid, (f) adding aroma volatiles obtained in step (b) to the concentrate, and (g) obtaining a substantially moisture free tea powder capable of being reconstituted in hot water to produce instant tea that is substantially free of haze and cloudiness. In this process, the aroma is added back to the concentrate which is free of insoluble matter, and the method relates to production of hot water soluble instant tea, which is known to be substantially free of hot water insoluble tea solids.

US2007/0160737 A1 (Unilever) discloses a method of manufacturing an aromatic green tea product for consumption as a beverage, comprising the steps of providing an aroma composition comprising t-2-hexenal and linalool at weight ratio of at least 0.7:1 and combining the aroma composition with the tea product.

### SUMMARY OF THE INVENTION

Botanical extracts of plants are widely used in cosmetic and pharmaceutical compositions. Often, they are used in combination, i.e., as separate or mixed extracts of two, three, four or even more plants. Most of the times, the extracts perform their own role, e.g., extract of the root of a plant X moisturizes the skin whereas extract of leaves of another plant Y increases the elasticity of skin. Very seldom are such extracts known to interact with each other to provide an altogether new effect or an effect which is more than the sum of their individual effects.

We have, under in vitro test conditions, unexpectedly observed synergistic interaction between the botanical extracts of a first plant Rubia cordifolia and a second plant Camellia sinensis The extracts interact synergistically to upregulate the markers associated with differentiation of keratinocytes, which in turn is closely linked with barrier properties of the epidermis. The markers, especially are loricrin and profilaggrin.

Disclosed in accordance with a first aspect of the invention is a topical composition comprising:
(i) a first extract comprising botanical actives extracted from Rubia cordifolia; and,
(ii) a second extract comprising volatile botanical actives extracted from Camellia sinensis,

wherein said second extract comprises E-2-hexenal and linalool at ratio of 0.1:1 to 10:1 parts by weight.

In accordance with a second aspect is disclosed the use of a topical composition of the first aspect for upregulation of markers associated with differentiation of keratinocytes.

In accordance with a third aspect is disclosed a method of upregulation of a marker associated with differentiation of keratinocytes, comprising a step of applying a topical cosmetic composition of the first aspect.

In accordance with a further aspect is disclosed a topical composition for use for the upregulation of a marker associated with differentiation of keratinocytes, said composition comprising:
(i) a first extract comprising botanical actives extracted from Rubia cordifolia; and,
(ii) a second extract comprising volatile botanical actives extracted from Camellia sinensis,

wherein said second extract comprises E-2-hexenal and linalool at ratio of 0.1:1 to 10:1 parts by weight.

### DETAILED DESCRIPTION

These and other aspects, features and advantages will become apparent to those of ordinary skill in the art from a reading of the following detailed description and the appended claims.

Numerical ranges expressed in the format “from x to y” are understood to include x and y. When for a specific feature multiple preferred ranges are described in the format “from x to y”, it is understood that all ranges combining the different end points are also contemplated.

As used herein the term “comprising” encompasses the terms “consisting essentially of” and “consisting of”. Where the term “comprising” is used, the listed steps or options need not be exhaustive. Unless otherwise specified, numerical ranges expressed in the format “from x to y” are understood to include x and y. In specifying any range of values or amounts, any particular upper value or amount can be associated with any particular lower value or amount. Except in the examples and comparative experiments, or where otherwise explicitly indicated, all numbers are to be understood as modified by the word “about”. All percentages and ratios contained herein are calculated by weight unless otherwise indicated. As used herein, the indefinite article “a” or “an” and its corresponding definite article “the” means at least one, or one or more, unless specified otherwise. The various features of the present invention referred to in individual sections above apply, as appropriate, to other sections mutatis mutandis. Consequently, features specified in one section may be combined with features specified in other sections as appropriate. Section headings are added for convenience only, and are not intended to limit the disclosure in any way.

The epidermis is composed of a variety of cells including keratinocytes, melanocytes and Langerhans cells. Keratinocytes constitute the major cells types forming about 75 to 90% of the total number of cells in the epidermis. Within the epidermis, the keratinocytes are usually found in one out of four distinct stages of differentiation.

The basal layer rests on the basal lamina separating the epidermis from the dermis. These are large columnar cells and they proliferate rapidly. These basal cells migrate upward within the epidermis due to the process of differentiation. The layer above the basal cells is the spinous layer.

The cells in the spinous layer initiate the production of proteins, characteristic of the differentiated epidermis. The granular layer, lying above the spinous layer, is characterized by electron-dense granules. This layer is responsible for synthesis of lipid molecules required for the formation of water-impermeable barrier of the skin. The topmost layer of the skin, the stratum corneum, is formed from the granular layer by destruction of cellular organelles. The cells in the stratum corneum called corneocytes contain extensively cross-linked proteins surrounded by a highly resistant cell envelope. The corneocytes are embedded in a layer of specific lipid structures and this structure acts like a protective barrier of the skin. The outermost layer of corneocytes gets peeled off from the skin during the normal process of desquamation. Differentiation of the epidermal keratinocytes is the driving force for normal desquamation. Epidermal differentiation is necessary for providing the essential protective environment in order to prevent loss of water from the skin and to maintain youthful appearance.

One of the main features of epidermal differentiation is the formation of an extensively cross-linked protein-enriched cornified envelope. Its formation is initiated in the differentiated upper layers of the epidermis by the cross-linking of specific protein substrates by a keratinocyte specific enzyme, transglutaminase-1 (TG-1). This enzyme is responsible for the glutamyl lysine cross-linking of these proteins into the insoluble cornified envelope. This process of differentiation also occurs in vitro in cultured keratinocytes (Refer: Thacher et al., Cell, (1985), pp. 685-695) when the cells are induced to differentiate in response to pro-differentiating agents.

During epidermal differentiation, keratinocytes undergo a well-defined series of morphological and biochemical changes in which actively proliferating basal cells differentiate stepwise through the spinous and granular cell layers to eventually form the anuclear squames characteristic of the protective stratum corneum at the skin surface.

Granular cells are characterised by the expression of profilaggrin. The profilaggrin gene encodes a high molecular weight phosphorylated polyprotein, composed of a number of related but non-identical filaggrin repeats.

The epidermal skin barrier is the natural protection layer of the body. The epidermal skin barrier forms an effective two-way protection barrier from the environment, preventing unwanted invasion of chemicals from outside, and preventing unregulated loss of water and nutrients from the inside.

Compositions in accordance with the invention comprise a first extract comprising botanical actives extracted from Rubia cordifolia and second extract comprising volatile botanical actives extracted from Camellia sinensis. The extracts are included as separate extracts, which in other words means that there is no co-extraction of the plant material. The extracts interact in a synergistic manner to upregulate certain markers of keratinocyte differentiation which are closely linked with the barrier function and the synergy is observed in in-vitro test conditions. The markers include at least one of loricrin and profilaggrin.

Rubia cordifolia

Rubia cordifolia Linn (Rubiaceae) is a perennial, herbaceous, slender, branched; climbing plant, with very long cylindrical roots, flexuous, with a thin red bark widely distributed in India, China, temperate Asia, Africa and tropical Australia. Rubia cordifolia has very high commercial and medicinal importance. Medicinally, Rubia cordifolia is important as the source of actives for various prescriptions in Ayurveda, traditional Chinese medicine system and other modern drugs. It is preferred that the botanical actives from Rubia cordifolia are extracted by subjecting its root, stem, leaves or bark to aqueous extraction, or the botanical actives are obtained from the phenolic fraction of the aqueous extract. In the present invention, it is further preferred that the first extract comprises botanical actives extracted from the stem of Rubia cordifolia. Its source and geographical origin is detailed hereinafter.

It is preferred that first extract comprises hydroxyanthraquinones and hydroxynapththoquinones which collectively account for 0.025 to 90% by weight of said first extract, more particularly 0.025 to 50% by weight and further more particularly 0.025 to 10% by weight. Further, in particular, it is preferred that the following process is used for extraction.

The stem of Rubia cordifolia is powdered and extracted with distilled water (1:10 by w/v) by refluxing for about 6 hours at 80° C. and then filtered through a cotton cloth. The filtrate is dried using a rota-evaporator at 45° C. under reduced pressure. A sticky powder is obtained which is termed as the botanical actives. This extract comprises collectively about 0.025 to 10% of hydroxyanthraquinones and hydroxynaphthoquinones by weight of the extract.

A preferred process for preparation of phenolic fraction of aqueous extract of Rubia cordifolia is as follows:

The aqueous extract (10 g), prepared as above is dissolved in 1 litre of 0.1 M NaOH solution. This solution is extracted with 1 litre ethyl acetate. The layer of ethyl acetate is separated from the mixture. The aqueous layer is neutralized with 1M HCl and then extracted with chloroform. The layer of chloroform is collected and washed with brine and dried using a rotary evaporator. The dry powder thus obtained is termed as the phenolic fraction of the aqueous extract. About 0.76 g of this fraction is obtained. This extract comprises collectively about 20 to 90% of hydroxyanthraquinones and hydroxynaphthoquinones by weight of the extract.

The phenolic fraction of botanical actives extracted from Rubia cordifolia comprise a mixture of hydroxyanthraquinones and hydroxynapththoquinones which preferably account for 0.025 to 90% by weight of said botanical actives.

Their individual amounts may vary but the collective amount is as indicated above.

The botanical actives are used as ingredients in the compositions of the invention. It is preferred that compositions in accordance with the invention comprise botanical actives extracted from Rubia cordifolia in an amount comprised in 0.001 to 10 wt % of the first extract. Such an extract is commercially available with a vendor named Phyto Life Sciences Pvt Ltd, India having their website at http://www.plpl.in/herbal-extract-india.htm

Camellia sinensis

Compositions of the present invention comprise volatile botanical extracts from Camellia sinensis which is the tea plant. The plant is Camellia sinensis var. sinensis. Alternatively, it is Camellia sinensis var. assamica. Further alternatively, the volatile botanical extracts are obtained from combination the above two varieties.

It is preferred that the volatile botanical actives from Camellia sinensis are extracted from its leaves or fibres, or said actives are comprised in fractions obtained by vacuum stripping, steam distillation or fractionation of the volatile botanical actives.

A preferred process for preparation of volatile botanical actives from Camellia sinensis is as follows:

The process comprises the first step of squeezing out the juice from fresh leaves of the plant. This step leaves behind residue of squeezed leaves. The residue is dried in a dryer. The step leads to dried leaf residue and a dryer exhaust which is rich in aromatic compounds. The aromatic compounds are recovered from the exhaust as aroma condensate. Further details of the process can be found in US2007/0160737 A1 (Unilever).

Preferably the aroma condensate is distilled to concentrate it. This aroma condensate is the volatile botanical actives extracted from Camellia sinensis. The actives comprise E-2-hexenal and linalool at a ratio of 0.1:1 to 10:1 parts by weight.

It is preferred that the ratio is 0.1:1 to 5:1. In addition to the two ingredients named above, the extract preferably comprises other volatile ingredients, namely methyl salicylate. The volatile extracts of Camellia sinensis are distinguished from various other extracts reported elsewhere by the particular ratio of the two important ingredients present therein. It is preferred that the volatile botanical actives extracted from Camellia sinensis comprise more than 80 wt % volatile ingredients, more preferably more than 90 wt % and yet more preferably more than 95 wt % volatile ingredients. A further point of distinction is that extracts reported elsewhere, would usually mean extracts containing substantial amount of non-volatile ingredients. Some other prior art compositions containing extracts of Camellia sinensis contain volatile as well as non-volatile ingredients in which one or both the ingredients are missing or the ratio is completely different.

It is preferred that the compositions according to the invention comprise volatile botanical actives extracted from Camellia sinensis in an amount comprised in 0.001 to 10 wt % of said second extract.

Further Details of the Composition of the Invention

“Topical composition” as used herein, is meant to include a composition for application to the external surface e.g. skin of mammals, especially humans for better skin health benefits. It is further preferred that the compositions of the invention is a cosmetic composition. Further preferably the compositions of the invention are leave-on or rinse-off compositions, preferably leave-on and includes any product applied to a human body primarily for enhanced health of skin but may be used also for improving the appearance, cleansing, odour control or general aesthetics. “Skin” as used herein is meant to include skin on the face and body (e.g., neck, chest, back, arms, underarms, hands, legs, buttocks and scalp) and especially to the under-eye portions of the face. The topical composition of the invention is especially useful for application on skin areas that get wrinkled or are more likely to get wrinkled especially to the sun exposed parts of the body.

The composition of the present invention could be in the form of a liquid, lotion, cream, foam, scrub, gel, soap bar or toner, or applied with an implement or via a face mask, pad or patch. Non-limiting examples of skin compositions include leave-on skin lotions and creams, shampoos, conditioners, shower gels, toilet bars, antiperspirants, deodorants, depilatories, lipsticks, foundations, mascara, sunless tanners and sunscreen lotions.

The compositions of the invention may be prepared according the usual manner of preparing such compositions. Reference may be made to standard text books and formulation guides.

When cosmetic composition is a cosmetic composition it preferably comprises a base preferably a dermatologically/cosmetically acceptable carrier. The carrier acts as a diluant, dispersant or carrier. The carrier may include materials commonly employed in skin compositions such as water, liquid or solid emollients, propellants, powders, emulsifiers, solvents, humectants, thickeners. The carrier in the compositions of the invention is single or mixtures of one or more vehicles. Preferably the carrier is present in the skin composition from 80 wt % to 99 wt %, preferably from 85 wt % to 90 wt %.

Compositions of the present invention will also include a cosmetically acceptable carrier. Water is the most preferred carrier. Amounts of water may, for example, range from 1 to 99%, preferably from 5 to 90%, more preferably from 35 to 70%, optimally between 40 and 60% by weight of the cosmetic composition. Ordinarily the compositions will be water and oil emulsions, which in some embodiments may be oil-in-water emulsions. Preferred emulsions are the water-in-oil variety.

Where the carrier is an emulsion, it is preferred that the particles are dispersed in the oil phase of the water and oil emulsion as this may improve the stability of the dye in the composition.

Emollient materials may be included as carriers in compositions of this invention.

These may be in the form of silicone oils, synthetic esters and/or hydrocarbons. Amounts of the emollients may range, for example, anywhere from 0.1 to 95%, more preferably between 1 and 50% by weight of the composition.

Silicone oils may be divided into the volatile and nonvolatile variety. The term “volatile” as used herein refers to those materials which have a measurable vapor pressure at ambient temperature (25° C.). Volatile silicone oils are preferably chosen from cyclic (cyclomethicone) or linear polydimethylsiloxanes containing from 3 to 9, preferably from 4 to 5, silicon atoms. In many liquid versions of compositions according to the present invention, the volatile silicone oils may form a relatively large component of the compositions as carriers. Amounts may range, for example, from 5% to 80%, more preferably from 20% to 70% by weight of the composition.

Nonvolatile silicone oils useful as an emollient material include polyalkyl siloxanes, polyalkylaryl siloxanes and polyether siloxane copolymers. The essentially nonvolatile polyalkyl siloxanes useful herein include, for example, polydimethyl siloxanes. Among the preferred nonvolatile emollients useful in the present compositions are the polydimethyl siloxanes.

Organopolysiloxane crosspolymers can be usefully employed. Representative of these materials are dimethicone/vinyl dimethicone crosspolymers and dimethicone crosspolymers available from a variety of suppliers including Dow Corning (9040, 9041, 9045, 9506 and 9509), General Electric (SFE 839), Shin Etsu (KSG-15, 16 and 18 [dimethicone/phenyl vinyl dimethicone crosspolymer]), and Grant Industries (Gransil® brand of materials), and lauryl dimethicone/vinyl dimethicone crosspolymers, all trademarked materials supplied by Shin Etsu (e.g. KSG-31, KSG-32, KSG-41, KSG-42, KSG-43 and KSG-44). Amounts of the aforementioned silicone elastomers (when present) will usually be from 0.1 to 20% by weight dissolved usually in a volatile silicone oil such as cyclomethicone.

When silicones are present in large amounts as carrier and water is also present, the systems may be oil continuous. These normally will require emulsification with a water-in-oil emulsifier such as a dimethicone copolyol (e.g. Abil® EM-90 which is cetyl dimethicone copolyol).

Among the ester emollients are:

a) Alkenyl or alkyl esters of fatty acids having 10 to 20 carbon atoms. Examples thereof include isoarachidyl neopentanoate, isodecyl neopentanoate, isononyl isonanoate, cetyl ricinoleate, oleyl myristate, oleyl stearate, and oleyl oleate.

b) Ether-esters such as fatty acid esters of ethoxylated fatty alcohols.

c) Polyhydric alcohol esters. Butylene glycol, ethylene glycol mono and di-fatty acid esters, diethylene glycol mono- and di-fatty acid esters, polyethylene glycol (200 to 6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl glycol esters of C1 to C30 alcohols. Exemplative is pentaerythrityl tetraethyl hexanoate.

d) Wax esters such as beeswax, spermaceti wax and tribehenin wax.

e) Sterols esters, of which cholesterol fatty acid esters are examples thereof.

f) Sugar ester of fatty acids such as sucrose polybehenate and sucrose polycottonseedate.

Of particular use also are the C.sub.12-15 alkyl benzoate esters sold under the Finsolve® brand.

Hydrocarbons which are suitable cosmetically acceptable carriers include petrolatum, mineral oil, C.sub.11-C.sub.13 isoparaffins, polyalphaolefins, and especially isohexadecane, available commercially as Permethyl® 101A from Presperse Inc.

Humectants of the polyhydric alcohol-type can be employed as cosmetically acceptable carriers. Typical polyhydric alcohols include polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1,2,6-hexanetriol, glycerol, ethoxylated glycerol, propoxylated glycerol and mixtures thereof. The amount of humectant may range, for example, anywhere from 0.5 to 50%, more preferably between 1 and 15% by weight of the composition. Most preferred is glycerol (also known as glycerin). Amounts of glycerin may range, for example, from 1% to 50%, more preferably from 10 to 35%, optimally from 15 to 30% by weight of the composition.

Besides cosmetically acceptable carriers, the compositions of this invention may include a variety of other functional ingredients. Sunscreen actives may be included in compositions of the present invention. These will be organic compounds having at least one chromophoric group absorbing within the ultraviolet ranging from 290 to 400 nm. Chromophoric organic sunscreen agents may be divided into the following categories (with specific examples) including: p-Aminobenzoic acid, its salts and its derivatives (ethyl, isobutyl, glyceryl esters; p-dimethylaminobenzoic acid); Anthranilates (o-aminobenzoates; methyl, menthyl, phenyl, benzyl, phenylethyl, linalyl, terpinyl, and cyclohexenyl esters); Salicylates (octyl, amyl, phenyl, benzyl, menthyl, glyceryl, and dipropyleneglycol esters); Cinnamic acid derivatives (menthyl and benzyl esters, alpha-phenyl cinnamonitrile; butyl cinnamoyl pyruvate); Dihydroxycinnamic acid derivatives (umbelliferone, methylumbelliferone, methylaceto-umbelliferone); Trihydroxycinnamic acid derivatives (esculetin, methylesculetin, daphnetin, and the glucosides, esculin and daphnin); Hydrocarbons (diphenylbutadiene, stilbene); Dibenzalacetone and benzalacetophenone; Naphtholsulfonates (sodium salts of 2-naphthol-3,6-disulfonic and of 2-naphthol-6,8-disulfonic acids); Dihydroxy-naphthoic acid and its salts; o- and p-Hydroxybiphenyldisulfonates; Coumarin derivatives (7-hydroxy, 7-methyl, 3-phenyl); Diazoles (2-acetyl-3-bromoindazole, phenyl benzoxazole, methyl naphthoxazole, various aryl benzothiazoles); Quinine salts (bisulfate, sulfate, chloride, oleate, and tannate); Quinoline derivatives (8-hydroxyquinoline salts, 2-phenylquinoline); Hydroxy- or methoxy-substituted benzophenones; Uric and vilouric acids; Tannic acid and its derivatives (e.g., hexaethylether); (Butyl carbityl) (6-propyl piperonyl) ether; Hydroquinone; Benzophenones (Oxybenzone, Sulisobenzone, Dioxybenzone, Benzoresorcinol, 2,2′,4,4′-Tetrahydroxybenzophenone, 2,2′-Dihydroxy-4,4′-d imethoxybenzophenone, Octabenzone; 4-Isopropyldibenzoylmethane; Butylmethoxydibenzoylmethane; Etocrylene; and 4-isopropyl-dibenzoylmethane). Particularly useful are: 2-ethylhexyl p-methoxycinnamate, 4,4′-t-butyl methoxydibenzoylmethane, 2-hydroxy-4-methoxybenzophenone, octyldimethyl p-aminobenzoic acid, digalloyltrioleate, 2,2-dihydroxy-4-methoxybenzophenone, ethyl 4-[bis(hydroxypropyl)]aminobenzoate, 2-ethylhexyl-2-cyano-3,3-diphenylacrylate, 2-ethylhexylsalicylate, glyceryl p-aminobenzoate, 3,3,5-trimethylcyclohexylsalicylate, methylanthranilate, p-dimethylaminobenzoic acid or aminobenzoate, 2-ethylhexyl p-dimethylaminobenzoate, 2-phenylbenzimidazole-5-sulfonic acid, 2-(p-dimethylaminophenyl)-5-sulfoniobenzoxazoic acid and mixtures thereof.

Particularly preferred are such materials as ethylhexyl p-methoxycinnamate, available as Parsol MCX®., Avobenzone, available as Parsol 1789®, Dermablock OS® (octylsalicylate) and Mexoryl SX® (with INCI name of Terephthalylidene Dicamphor Sulfonic Acid). Amounts of the organic sunscreen agent may range, for example, from 0.1 to 15%, more preferably from 0.5% to 10%, optimally from 1% to 8% by weight of the composition.

A variety of thickening agents may be included in the compositions. Illustrative but not limiting are stearic acid, Acrylamide/Sodium Acryloyldimethyltaurate Copolymer (Aristoflex AVC®), Hydroxyethyl Acrylate/Sodium Acryloyldimethyltaurate Copolymer, Aluminum Starch Octenyl Succinate, Polyacrylates (such as Carbomers including Carbopol® 980, Carbopol® 1342, Pemulen TR-2® and the Ultrez® thickeners), Polysaccharides (including xanthan gum, guar gum, pectin, carageenan and sclerotium gums), celluloses (including carboxymethyl cellulose, ethyl cellulose, hydroxyethyl cellulose and methyl hydroxymethyl cellulose), minerals (including talc, silica, alumina, mica and clays, the latter being represented by bentonites, hectorites and attapulgites), magnesium aluminum silicate and mixtures thereof. Amounts of the thickeners may range, for example, from 0.05 to 10%, more preferably from 0.3 to 2% by weight of the composition.

Preservatives can desirably be incorporated into the cosmetic compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are phenoxyethanol, methyl paraben, propyl paraben, butyl paraben, isobutyl paraben, imidazolidinyl urea, sodium dehydroacetate and benzyl alcohol. The preservatives should be selected having regard for the use of the composition and possible incompatibilities between the preservatives and other ingredients in the composition. Preservatives are preferably employed in amounts ranging from 0.01% to 2% by weight of the composition.

Compositions of the present invention may also contain vitamins and flavonoids. Illustrative water-soluble vitamins are Niacinamide, Vitamin B2, Vitamin B6, Vitamin C and Biotin. Among the useful water-insoluble vitamins are Vitamin A (retinol), Vitamin A Palmitate, ascorbyl tetraisopalmitate, Vitamin E (tocopherol), Vitamin E Acetate and DL-panthenol. A particularly suitable Vitamin B6 derivative is Pyridoxine Palmitate. Among the preferred flavonoids are glucosyl hesperidin and rutin. Total amount of vitamins or flavonoids when present in compositions according to the present invention may range, for example, from 0.001 to 10%, more preferably from 0.01% to 1%, optimally from 0.1 to 0.5% by weight of the composition.

Desquamation agents are further optional components. Illustrative are the alpha-hydroxycarboxylic acids and beta-hydroxycarboxylic acids and salts of these acids. Among the former are salts of glycolic acid, lactic acid and malic acid. Salicylic acid is representative of the beta-hydroxycarboxylic acids. Amounts of these materials when present may range from 0.1 to 15% by weight of the composition.

A variety of herbal extracts may optionally be included in compositions of this invention. Illustrative are pomegranate, white birch (Betula Alba), green tea, chamomile, licorice, boswellia serrata, olive (Olea Europaea) leaf, arnica montana flower, Lavandula angustifolia, and extract combinations thereof. The extracts may either be water soluble or water-insoluble carried in a solvent which respectively is hydrophilic or hydrophobic. Water and ethanol are the preferred extract solvents.

Miscellaneous other adjunct cosmetic ingredients that may be suitable for the present compositions include ceramides (e.g. Ceramide 3 and Ceramide 6), conjugated linoleic acids, colorants (e.g. iron oxides), metal (manganese, copper and/or zinc) gluconates, allantoin, palmitoyl pentapeptide-3, amino acids (e.g. alanine, arginine, glycine, lysine, proline, serine, threonine, glumatic acid and mixtures thereof), trimethylglycine, sodium PCA, chelator like disodium EDTA, opacifiers like titanium dioxide, magnesium aspartate, and combinations thereof. Amounts may, for example, vary from 0.000001 to 3% by weight of the composition.

A small amount of emulsifying surfactant may be present. Surfactants may be anionic, nonionic, cationic, amphoteric and mixtures thereof. Levels may range, for example, from 0.1 to 5%, more preferably from 0.1 to 2%, optimally from 0.1 to 1% by weight.

Advantageously the amount of surfactant present should not be sufficient for lather formation. In these instances, less than 2% by weight, preferably less than 1%, and optimally less than 0.5% by weight surfactant is present. Emulsifiers like PEG-100 stearate may be used as well as emulsion stabilizers like cetearyl alcohol and ceteareth-20 may be used and typically in amounts that do not exceed 5 percent by weight of the composition.

Other optional additives suitable for use in the composition of this invention include cationic ammonium compounds to enhance moisturization. Such compounds include salts of hydroxypropyltri (C1 to C3 alkyl) ammonium mono-substituted-saccharide, salts of hydroxypropyltri (C1 to C3 alkyl) ammonium mono-substituted polyols, dihydroxypropyltri (C1 to C3 alkyl) ammonium salts, dihydroxypropyldi (C1 to C3 alkyl) mono(hydroxyethyl) ammonium salts, guar hydroxypropyl trimonium salts, 2,3-dihydroxypropyl tri(C1 to C3 alkyl or hydroxalkyl) ammonium salts or mixtures thereof. In a most preferred embodiment and when desired, the cationic ammonium compound employed in this invention is the quaternary ammonium compound 1,2-dihydroxypropyltrimonium chloride. If used, such compounds typically make up from 0.01 to 30%, and more preferably from about 0.1 to about 15% by weight of the composition.

When cationic ammonium compounds are used, optional additives for use with the same are moisturizing agents such as substituted ureas like hydroxymethyl urea, hydroxyethyl urea, hydroxypropyl urea; bis(hydroxymethyl) urea; bis(hydroxyethyl) urea; bis(hydroxypropyl) urea; N,N′-dihydroxymethyl urea; N,N′-di-hydroxyethyl urea; N,N′-di-hydroxypropyl urea; N,N,N′-tri-hydroxyethyl urea; tetra(hydroxymethyl) urea; tetra(hydroxyethyl) urea; tetra(hydroxypropyl) urea; N-methyl-N′-hydroxyethyl urea; N-ethyl-N′-hydroxyethyl urea; N-hydroxypropyl-N′-hydroxyethyl urea and N,N′dimethyl-N-hydroxyethyl urea or mixtures thereof. Where the term hydroxypropyl appears, the meaning is generic for either 3-hydroxy-n-propyl, 2-hydroxy-n-propyl, 3-hydroxy-i-propyl or 2-hydroxy-i-propyl radicals. Most preferred is hydroxyethyl urea. The latter is available as a 50% aqueous liquid from the National Starch & Chemical Division of ICI under the trademark Hydrovance®. Such substituted ureas, while desirable in moisturizing formulations, are only selected for use when compatible with sunless tanning agent or agents (if present) used in the compositions of this invention.

Amounts of substituted urea, when used, in the composition of this invention range from 0.01 to 20%, more preferably from 0.5 to 15%, and most preferably from 2 to 10% based on total weight of the composition and including all ranges subsumed therein.

When cationic ammonium compound and substituted urea are used, in a most especially preferred embodiment at least from 0.01 to 25%, more preferably from 0.2 to 20%, and most preferably from 1 to 15% humectant, like glycerine, is used, based on total weight of the composition and including all ranges subsumed therein.

When making the compositions of this invention, ingredients are typically mixed with moderate shear under atmospheric conditions. Preferably, the compositions display a pH from 4 to 6.

Packaging for the composition of this invention can be a jar or tube as well as any other format typically seen for cosmetic, cream, washing and lotion type products. The compositions may be applied topically and preferably 1-4 milligrams of composition is applied per square centimeter of skin. The composition is preferably substantially white or colourless (transparent) when in packaged form but is transformable to a coloured composition on application to skin.

Method and Use According to the Invention

In one aspect the use is non-therapeutic in nature. Alternatively, it is therapeutic in nature.

When the use is non-therapeutic in nature, it preferably is for cosmetic purpose.

In one aspect the method of the invention is non-therapeutic in nature. Alternatively, it is therapeutic in nature. When the method is non-therapeutic in nature, it preferably is for cosmetic purpose.

The description of preferred aspects pertaining to the use of the composition applies mutatis mutandis to the method of the invention.

The method of the present invention may be carried out one or more times daily by applying to the skin which requires improvement epidermal skin barrier benefits. The amount of the composition used and the frequency with which it is applied may vary. In general, a small quantity of the composition, for example from 0.1 to 5 ml is applied to the skin. A rinsing step may optionally follow depending on whether the composition is formulated as a “leave-on” or a “rinse-off” product.

Now the invention will be demonstrated by means of following non-limiting examples.

### EXAMPLES

### Example 1

The source of the aqueous extract of the stem of Rubia cordifolia was Phyto LifeSciences P. Ltd. Ahmedabad, India. It is commercially available with the vendor. The geographical origin of Rubia cordifolia was India. Before use, the extract was diluted to 0.001% with the cell culture media for keratinocytes referred below.

Volatile botanical extracts of Camellia sinensis were obtained in-house by following the method disclosed in US2007/0160737 A1 (Unilever). The geographical origin of the of the plant was India. The extract contained E-2-hexenal and linalool at a ratio of 0.14:1 parts by weight. Before use, the extract was diluted to 0.001% using the same cell culture medium as abovementioned.

1*10\hat{ }5 HEKa (Human epidermal adult keratinocytes) were plated in a 12-well plate and incubated at 37° C. in 5% CO2 for 24 hours. Both the actives were added the next day and cells were harvested after 24 hours. Total cellular RNA was extracted from these cells using Ambion Purelink RNA mini Kit (CAT #12183020). The cDNA synthesis was carried out using Bio-Rad iScript (CAT #1708891). q PCR was then carried out using specific forward and reverse primers as listed below in Table 1. The PCR was carried out in Bio-Rad DNA engine gradient cycler system for 40 cycles in our case. The fold change was calculated using ΔΔcT method. (Reference WO 2010/046316 A2, Unilever).

TABLE 1

| Genes | Forward (5′-3′) | Reverse (5′-3′) |
|---|---|---|
| Loricrin | CAAACCTCGG | ACCTGGCCGT |
| | GTAGCATCAT | CCAAATAGAT |
| Profilaggrin | ATCACAGCCA | GTCTCCGACTG |
| | CACCACATC | TTCCTCATTAC |

The fold change with respect to control was 2^{-(ΔcT)}.

The data of gene expression is summarised in Table 2. The data indicates whether there was up or down regulation of profillagrin and loricrin and the extent of the regulation.

TABLE 2

| | Loricrin/ | | | Profillagrin/ | | |
|---|---|---|---|---|---|---|
| | Fold | | | Fold | | |
| | Change | | | Change | | |
| Details | N1 | N2 | SD | N1 | N2 | SD |
| Control (no extract only 1 | 1.00 | 1.00 | 0.0 | 1.00 | 1.00 | 0.0 |
| ml medium) | | | | | | |
| Botanical extracts of Rubia | 1.46 | 0.57 | 0.6 | 0.66 | 0.32 | 0.2 |
| cordifolia (1 ml medium | | | | | | |
| containing 500 ppm of the | | | | | | |
| extract) in each well | | | | | | |
| Volatile botanical extracts | 1.34 | 1.59 | 0.2 | 0.67 | 0.91 | 0.2 |
| of Camellia sinensis (1 ml of | | | | | | |
| medium containing 10 ppm | | | | | | |
| of the extract) in each well | | | | | | |
| 1 ml of medium containing | 33.72 | 13.35* | 14.4 | 4.95 | 7.42 | 1.7 |
| 10 ppm of Botanical actives | | | | | | |
| of Camellia sinensis and 500 | | | | | | |
| ppm of botanical extracts of | | | | | | |
| Rubia cordifolia in each well | | | | | | |

Note:
The average value of N1 and N2 should be considered
SD means Standard Deviation

The data in Table 2 indicates upregulation of profillagrin and loricrin in the experiment where botanical extracts of Rubia cordifolia and volatile botanical extracts of Camellia sinensis were together. The data is an indicator of enhancement in differentiation of keratinocytes, which in turn indicates that the combination of actives provide epidermal skin barrier benefits.

1. A topical composition comprising:
(i) a first extract comprising botanical actives extracted from Rubia cordifolia; and,
(ii) a second extract comprising volatile botanical actives extracted from Camellia sinensis,
wherein said second extract comprises E-2-hexenal and linalool at ratio of 0.1:1 to 10:1 parts by weight.

2. A composition as claimed in claim 1 wherein said ratio is from 0.1:1 to 5:1 parts by weight.

3. A topical composition as claimed in claim 1 wherein said botanical actives from Rubia cordifolia are extracted from its root, stem, leaves or bark by hydro-alcoholic extraction.

4. A composition as claimed in claim 3 wherein said first extract comprises hydroxyanthraquinones and hydroxynapththoquinones which collectively account for 0.025 to 90% by weight of said first extract.

5. A topical composition as claimed in claim 1 wherein said volatile botanical actives from Camellia sinensis are extracted from its leaves or fibres, or said actives are comprised in fractions obtained by vacuum stripping, steam distillation or fractionation of said volatile botanical actives.

6. A topical composition as claimed in claim 1 wherein said composition is a cosmetic composition.

7. A topical composition as claimed in claim 1 comprising botanical actives extracted from Rubia cordifolia in an amount comprised in 0.001 to 10 wt % of said first extract.

8. A topical composition as claimed in claim 1 comprising volatile botanical actives extracted from Camellia sinensis in an amount comprised in 0.001 to 10 wt % of said second extract.

9. Use of a topical composition as claimed in of claim 1 for upregulation of markers associated with differentiation of keratinocytes.

10. Use as claimed in claim 9 wherein said marker is at least one of loricrin or profillagrin.

11. Use as claimed in claim 9 wherein said use is for non-therapeutic purpose.

12. Use as claimed in claim 9 wherein said differentiation of keratinocytes leads to epidermal skin barrier benefits.

13. A method of upregulation of a marker associated with differentiation of keratinocytes, comprising a step of applying a topical cosmetic composition as claimed in claim 1.

14. A topical composition for use for the upregulation of a marker associated with differentiation of keratinocytes, said composition comprising:
(iii) a first extract comprising botanical actives extracted from Rubia cordifolia; and,
(iv) a second extract comprising volatile botanical actives extracted from Camellia sinensis,
wherein said second extract comprises E-2-hexenal and linalool at ratio of 0.1:1 to 10:1 parts by weight.

15. Use of a first extract comprising botanical actives extracted from Rubia cordifolia and a second extract comprising volatile botanical actives extracted from Camellia sinensis in the manufacture of a topical composition for upregulation of a marker associated with differentiation of keratinocytes, wherein said second extract comprises E-2-hexenal and linalool at ratio of 0.1:1 to 10:1 parts by weight of the extract.
